(19) United States
(12) Patent Application Publication
(10) Pub. No.: US 2022/0304946 A1
(43) Pub. Date: Sep. 29, 2022

(54) TRANS-ANETHOLE ((E)-1-METHOXY-4- (1-PROPENYL) BENZENE), A NEW AND POTENT INHIBITOR OF PROLYL ENDOPEPTIDASE

(71) Applicants: Muhammad Imran Khan, Islamabad (PK); Depeng Wang, Linyl (CN); Najeeb Ullah, Peshawa (PK); Fawad Ali Shah, Islamabad (PK); Shah Fahad, Haripur (PK); Shahid Ullah Khan, Guanshan (CN); Abdul Wahab, Kohat (PK); Arif Ullah Khan, Islamabad (PK)

(72) Inventors: Muhammad Imran Khan, Islamabad (PK); Depeng Wang, Linyl (CN); Najeeb Ullah, Peshawa (PK); Fawad Ali Shah, Islamabad (PK); Shah Fahad, Haripur (PK); Shahid Ullah Khan, Guanshan (CN); Abdul Wahab, Kohat (PK); Arif Ullah Khan, Islamabad (PK)

(21) Appl. No.: 17/214,886

(22) Filed: Mar. 28, 2021

(51) Int. Cl. A61K 31/085; A61K 45/06

(52) U.S. Cl. CPC A61K 31/085; A61K 45/06

(57) ABSTRACT

Overexpression of prolyl endopeptidase (PEP) activity in the brain was found to be linked with neurodegenerative disorders, and the memory loss caused by amnesic compounds can be restored by PEP inhibitors. In the current intervention, the PEP inhibitory activity of trans-anethole ((E)-1-Methoxy-4-(1-propenyl)benzene), an essential oil that is naturally occurring in Illicium verum (star anise) has been assessed. More specifically, the present invention relates to finding of a potent inhibitor of prolyl endopeptidase (Trans-Anethole ((E)-1-Methoxy-4-(1-propenyl)benzene).

### CROSS-REFERENCE TO RELATED APPLICATIONS

N/A

### BACKGROUND OF THE INVENTION

### 1. Field of the Invention

The present invention relates to a novel pharmacological activity of trans-anethole ((E)-1-Methoxy-4-(1-propenyl) benzene) derived from Illicium verum (star anise) as a potent prolyl endopeptidase (PEP) inhibitor, which has not been reported before.

### 2. Description of Related Art

Prolyl endopeptidase (prolyl oligopeptidase, PEP, POP, EC 3.4.21.26) is a large intracellular serine protease, and cause cleavage of the short-length peptides (<30 amino acids) at the carboxyl side of an internal proline. PEP has been present in various organs, mostly in the brain of patients affected with amnestic disorders. PEP considerably contributes to degrading biologically active peptides, neuropeptides, and hormones comprising of proline residues including vasopressin, oxytocin, neurotensin, Substance-P, and angiotensin that have been associated with many neurological disorders, e.g. amnesia, Alzheimer's disease, schizophrenia, and depression. A reported study revealed an elevated expression level of PEP in the hippocampus of transgenic mice (adult) prior to the formation of β-amyloid plaques while at the same time memory deficits were developed. It has also been reported that Altered serum PEP activity has a key role in numerous psychiatric disorders, and abnormal levels of PEP activity is considerably elevated in the brains of patients affected with Alzheimer relative to normal individuals. Hence, PEP inhibitors can be used as therapeutic agents for progressive memory deficits and cognitive dysfunction associated with ageing and neurodegenerative diseases of the central nervous system.

In recent decades, numerous inhibitors of PEP have been developed, that may treat many clinical conditions of the brain, as shown by the neuroprotective and cognition-enhancing effects of PEP inhibitors in various animal models. There are many inhibitors of PEP, such as baicalin, pramiracetam, KYP-2047, JTP-4819, and S-17092. It has been reported that most of the inhibitors are synthetic in nature and substrate-like which are based on the structure of N-acyl-L-prolyl-pyrrolidine. However, some studies reported PEP inhibitors from natural sources. The chemical diversity of natural compounds is unique and the underlined compounds having optimal binding with biological macromolecules. Hence, so far, natural products have been proved to be the richest source of novel compound classes for biological studies that can lead towards the discovery of new drugs. For instance, from 2005 to 2007, a total of 13 natural product and drugs derived from the natural product were approved across the globe including exenatide, ziconotide, and ixabepilone which are the first members of new human drug classes. The underlined evidence supports the significance of natural products in the discovery of drugs.

In view of the above application of natural products, the present invention was made to find the in-vitro PEP inhibitory activity of (E)-1-Methoxy-4-(1-propenyl) benzene (trans-anethole). This compound has several biological activities that have been reported in the literature including anti-metastatic, anti-oxidative, antimicrobial, antiviral, and anti-inflammatory activities. It has also been shown that trans-anethole can modify the functions of Ca⁺² and Ca⁺²-activated K⁺ channels.

### BRIEF SUMMARY OF THE INVENTION

The present invention relates to the prolyl endopeptidase (PEP) inhibitory activity of trans-anethole ((E)-1-Methoxy-4-(1-propenyl) benzene), an essential oil that is naturally occurring in Illicium verum (star anise). The underlined compound was tested for PEP inhibitory activity and cytotoxicity against mouse fibroblast 3T3 cell lines. Kinetic assay of Trans anethole was carried out to find the mechanism of inhibition of PEP against the standard inhibitor i.e., bacitracin. The results showed that the underlined natural compound has potent PEP inhibitory activity with IC50=5.70±1.20 μM when compared with the standard inhibitor i.e., bacitracin (IC50=114.00±1.20 μM). Besides, 10 to 500 μM concentration of trans-anethole, showed no obvious effect on the growth and proliferation of 3T3 cell of mouse fibroblasts. Therefore, trans-anethole was found as an effective natural drug candidate that could be used against neurodegenerative disorders, such as dementia and Alzheimer's disease (AD).

### BRIEF DESCRIPTION OF THE DRAWING FIGURES

FIG. 1 structure of (E)-1-Methoxy-4-(1-propenyl)benzene, and its PEP inhibitory potential (IC50).

FIG. 2A Lineweaver-Burk plot i.e. reciprocal rate of reaction vs reciprocal of the substrate (Z-GlypNA) in the absence (▪) and the presence of 24.00 μM (○), 12.00 μM (●), 6.00 μM (□), 3.00 μM (○), 1.50 μM (●), and 0.00 μM (●) of trans-anethole.

FIG. 2B shows the double reciprocal plot for evaluating Ki and is between slope and concentration of inhibitor.

FIG. 2C indicates the Dixon plot between 1/Vmax and trans-anethole, which further confirmed that it competitively inhibited PEP catalytic activity.

### DETAILED DESCRIPTION OF THE INVENTION

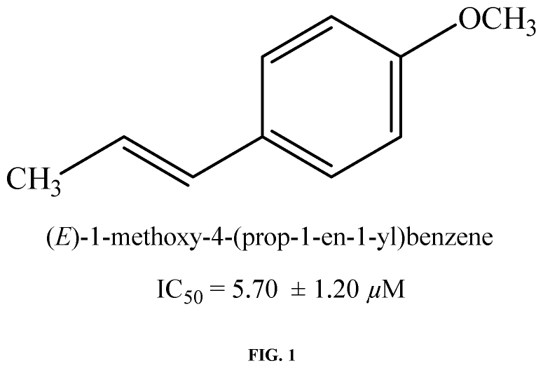

The present invention is valuable as a potential natural drug candidate against neurodegenerative disorders, such as dementia and Alzheimer's disease (AD). Overexpression of prolyl endopeptidase (PEP) activity in the brain was found to be linked with neurodegenerative disorders, and the memory loss caused by amnesic compounds can be restored by PEP inhibitors. Herein, the current study assessed the PEP inhibitory activity of (E)-1-Methoxy-4-(1-propenyl) (benzene trans-anethole, FIG. 1), an essential oil that is naturally occurring in Illicium verum (star anise).

PEP inhibitory activity was evaluated with minor modification of the Yoshimoto et al. method. Briefly, 150 μL of sodium phosphate buffer (50 mM, pH 7.0), and 10 μL of the test compound (0.5 mM in DMSO) were added into a 96-well plate, followed by adding 20 μL solution containing 0.02 units of PEP enzyme. In the Blank reaction, 20 μL of DMSO was added instead of the test compound, while bacitracin (0.5 mM) was used as the positive control. The incubation of the reaction mixture was carried out for 15 min at 30° C. after which a pre-read was taken at 410 nm. Next, 0.4 mM solution of Z-Gly-Pro-pNA was formed in aqueous 1,4-dioxane (40%), followed by taking the optical density (O.D.) of the reaction mixture at a wavelength of 410 nm for 40 min using a 96-well plate reader (SpectraMax-384, Molecular Devices, CA, USA).

In order to assess the inhibitory mechanism of trans-anethole. That in which way it inhibits the PEP catalytic activity i.e. competitive inhibition, non-competitive inhibition, mixed or uncompetitive inhibition. The kinetic assay was carried out by incubation of PEP (0.02 units/200 μL) for 15 min at 30° C. with varying concentrations of trans-anethole. The reaction was started post adding four various concentrations of Z-Gly-Pro-pNA (0.2 mM to 0.5 mM). The PEP enzymatic catalysis was assessed at 410 nm via a 96-well plate reader.

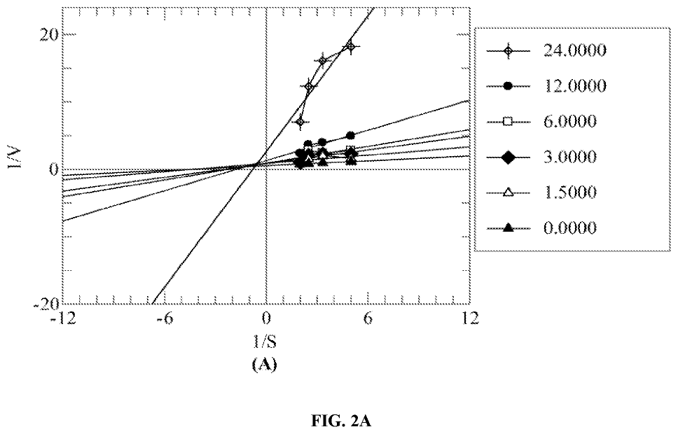

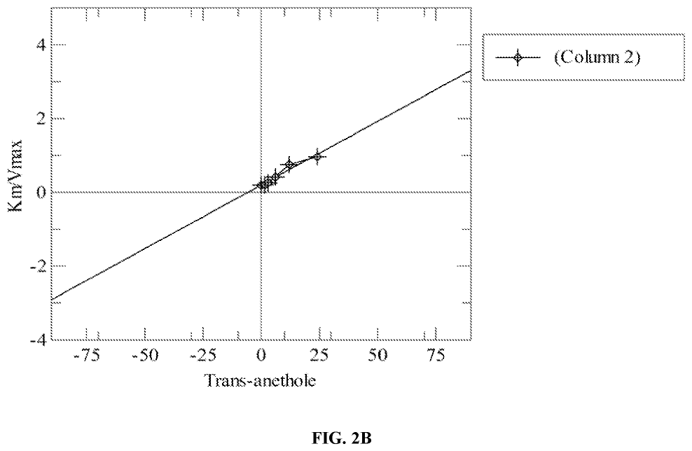

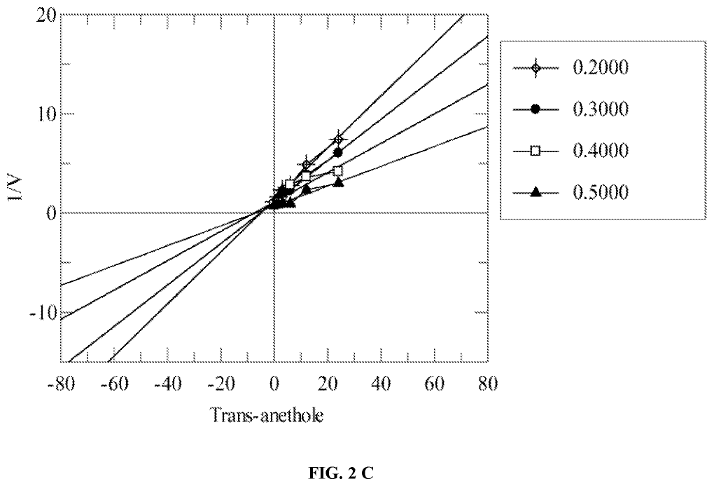

Next, using the obtained O.D. results, the Lineweaver-Burk and Dixon plot was generated. The dissociation constant i.e., Ki value of trans-anethole was calculated by Lineweaver-Burk plot with various concentration of trans-anethole, followed by reconfirmation of the Ki value by Dixon plot, in which the reciprocal rate of reaction and different trans-anethole concentrations were plotted against each other (FIG. 2A-C).

The cytotoxicity of trans-anethole was examined by using the standard MTT (thiazolyl blue tetrazolium bromide) colourimetric assay, which indicates cellular metabolic activity. For this purpose, 3T3 cells of mouse fibroblast cell lines were grown in each well (at a density of 3.5×104 per well). The plates were then placed at 30° C. in a 5% CO2 incubator for 48 hrs, followed by treating with sample solutions (10-500 μM). Next, the addition of MTT solution (200 μL, 0.5 mg/mL) was carried out into each well and the plate was left for second incubation (at 30° C. for 4 hrs). Using a microplate reader (SpectraMax-384, USA), the extent of MTT reduction to formazan cells was measured by recording the O.D. at 540 nm. Cytotoxic concentration that inhibited 50% growth was recorded as IC50 of 3T3 cells.

The IC50 value of trans-anethole was determined by EZ-FIT (Perrella Scientific, Inc., USA). Each reaction was repeated thrice and the differences in the results of each experiment were collectively expressed as the standard error of the mean (SEM). The underlined calculations were used for obtaining the percent inhibition of trans-anethole.

% Inhibition=100−(O.D. test compound/O.D. Control)×100

Grafit version-7 (Erithacus Software Limited, UK) was employed for the Kinetic studies.

Trans-anethole ((E)-1-Methoxy-4-(1-propenyl)benzene), which is reported in the literature as anti-metastatic activity, anti-oxidative, antimicrobial and antiviral, anti-inflammatory properties. It has also been shown that trans-anethole can modify Ca⁺² and Ca⁺²-activated K′ channels function. Herein, this study revealed the PEP enzyme inhibitory activity and cytotoxicity studies of trans-anethole. On the basis of obtained results, the underlined compound showed a potent PEP inhibitory activity with an IC50 value of 5.70±1.20 μM. The standard inhibitor i.e., bacitracin has several time higher IC50 value (IC50=114.00±1.20 μM) than that of trans-anethole IC50. According to the literature survey, for the first time, the current study revealing the PEP inhibitory activity of the underlined compound.

According to the cytotoxicity studies, the presence of 500 μM trans-anethole concentration showed no obvious effects on the growth as well as the proliferation of mouse fibroblasts 3T3 cell lines.

The kinetic studies suggested that trans-anethole is a competitive type of PEP enzyme inhibitor with a Ki value of 5.656±0.03 04. FIG. 2A indicates the Lineweaver-Burk plot of trans-anethole, in which the x-axis indicates 1/S (inverse of substrate i.e., Z-Gly-pro-pNA concentrations), while the y-axis shows 1/V (inverse of Vmax values). In the absence (i.e., 00.00 μM) and the presence of 35.00, 17.50, and 8.75 μM of trans-anethole. The figure shows that apparent Km increases while Vmax remains unchanged. In this view, it is a competitive-type PEP inhibitor. FIG. 2B shows secondary replot i.e. reciprocal of slope vs various concentrations of trans-anethole and FIG. 2C shows Dixon plot i.e. reciprocal of rate of reaction vs various concentrations of trans-anethole. It seems that this compound has possible sites for interaction with the catalytic amino acid residues at the active site, results in the prevention of PEP catalytic activity.

1. A new prolyl endopeptidase (PEP) inhibitory activity potential of Trans-Anethole ((E)-1-Methoxy-4-(1-propenyl)benzene).

2. The compound as claimed in claim 1) show potent PEP inhibitory activity with IC50=5.70±1.20 μM when compared with the standard inhibitor i.e., Bacitracin (IC50=114.00±1.20 μM).

3. The compound as claimed in claim 1) show no obvious effect on the growth and proliferation of 3T3 cell of mouse fibroblasts when tested in concentration range of 10 to 500 μM.

4. A method for the treatment of neurodegenerative disorders, such as dementia and Alzheimer's disease (AD), comprising of administration of a therapeutically effective amount of Trans-Anethole ((E)-1-Methoxy-4-(1-propenyl)benzene) to humans and animals.

5. As claimed in claim 4, where the said Trans-Anethole ((E)-1-Methoxy-4-(1-propenyl)benzene) is administered in a pharmaceutically elegant dosage form.

6. A pharmaceutical composition, which comprises of an effective quantity of Trans-Anethole ((E)-1-Methoxy-4-(1-propenyl)benzene) and a pharmaceutically acceptable vehicle for administration to humans and animals for the treatment of neurodegenerative disorders, such as dementia and Alzheimer's disease (AD).

7. A pharmaceutical composition as claimed in claim 6 wherein it is combined with other known anti-neurodegenerative drugs.
